SEQUENCE LISTING (22 sequences)

SEQ ID NO: 1 - DNA, 20 bp, Artificial Sequence
  Forward primer for Treponema denticola
ccttgaacaaaaaccggaaa

SEQ ID NO: 2 - DNA, 20 bp, Artificial Sequence
  Reverse primer for Treponema denticola
gggaaaagcaggaagcataa

SEQ ID NO: 3 - DNA, 23 bp, Artificial Sequence
  Forward primer for Aggregatibacter actinomycetemcomitans
cttacctactcttgacatccgaa

SEQ ID NO: 4 - DNA, 21 bp, Artificial Sequence
  Reverse primer for Aggregatibacter actinomycetemcomitans
atgcagcacctgtctcaaagc

SEQ ID NO: 5 - DNA, 20 bp, Artificial Sequence
  Forward primer for Porphyromonas gingivalis
tggtttcatgcagcttcttt

SEQ ID NO: 6 - DNA, 20 bp, Artificial Sequence
  Reverse primer for Porphyromonas gingivalis
tcggcaccttcgtaattctt

SEQ ID NO: 7 - DNA, 23 bp, Artificial Sequence
  Forward primer for Tannerella forsythia
gataggcttaacacatgcaagtc

SEQ ID NO: 8 - DNA, 20 bp, Artificial Sequence
  Reverse primer for Tannerella forsythia
gttgcgggcaggttacatac

SEQ ID NO: 9 - DNA, 22 bp, Artificial Sequence
  Forward primer for Fusobacterium nucleatum
aagcgcgtctaggtggttatgt

SEQ ID NO: 10 - DNA, 23 bp, Artificial Sequence
  Reverse primer for Fusobacterium nucleatum
tgtagttccgcttacctctccag

SEQ ID NO: 11 - DNA, 26 bp, Artificial Sequence
  Forward primer for Peptostreptococcus micros
aaacgacgattaataccacatgagac

SEQ ID NO: 12 - DNA, 19 bp, Artificial Sequence
  Reverse primer for Peptostreptococcus micros
actgctgcctcccgtagga

SEQ ID NO: 13 - DNA, 21 bp, Artificial Sequence
  Forward primer for Prevotella intermedia
tccaccgatgaatctttggtc

SEQ ID NO: 14 - DNA, 20 bp, Artificial Sequence
  Reverse primer for Prevotella intermedia
atccaaccttccctccactc

SEQ ID NO: 15 - DNA, 25 bp, Artificial Sequence
  Forward primer for Candida albicans
gccggtgacgacgctccaagagctg

SEQ ID NO: 16 - DNA, 27 bp, Artificial Sequence
  Reverse primer for Candida albicans
ccgtgttcaattgggtatctcaaggtg

SEQ ID NO: 17 - DNA, 25 bp, Artificial Sequence
  Forward primer for Streptococcus mutans
gcctacagctcagagatgctattct

SEQ ID NO: 18 - DNA, 23 bp, Artificial Sequence
  Reverse primer for Streptococcus mutans
gccatacaccactcatgaattga

SEQ ID NO: 19 - DNA, 19 bp, Artificial Sequence
  Forward primer for Capnocytophaga species
aagtcgagggagaagccct

SEQ ID NO: 20 - DNA, 22 bp, Artificial Sequence
  Reverse primer for Capnocytophaga species
tccaaatttcttcggggctatc

SEQ ID NO: 21 - DNA, 19 bp, Artificial Sequence
  Universal forward primer
tcctacgggaggcagcagt

SEQ ID NO: 22 - DNA, 26 bp, Artificial Sequence
  Universal reverse primer
ggactaccagggtatctaatcctgtt

(19) United States
(12) Patent Application Publication
(10) Pub. No.: US 2018/0230519 A1
(43) Pub. Date: Aug. 16, 2018

(54) METHODS AND KITS FOR SELECTING TREATMENT FOR ORAL INFECTIONS

(71) Applicant: OraVital Inc., Toronto (CA)

(72) Inventors: Anne Bosy, Etobicoke (CA); James Hyland, Toronto (CA)

(73) Assignee: OraVital Inc., Toronto (CA)

(21) Appl. No.: 15/434,018

(22) Filed: Feb. 15, 2017

(51) Int. Cl. C12Q 1/68; A61K 31/4164; A61K 31/43; A61K 31/7048; A61K 31/65; A61K 31/138; A61B 10/00

(52) U.S. Cl. CPC C12Q 1/689; C12Q 1/6895; A61K 31/4164; A61K 31/43; C12Q 2600/158; A61K 31/65; A61K 31/138; A61B 10/0051; A61K 31/7048

(57) ABSTRACT

The present disclosure describes methods and kits for selecting treatments for, and treating, an oral infection in a patient. Oral samples are collected from multiple locations of the patient's oral cavity and include saliva, tongue biofilm, and interproximal biofilm. The samples are analyzed to determine a pathogen profile of the oral cavity, using nucleic-acid based analysis. Based on the pathogen profile, an antibiotic rinse is selected as the treatment of the oral infection.

### TECHNICAL FIELD

The technical field relates to methods and kits for identifying treatments for, and treating, oral infections, including periodontal diseases, dental caries, endodontitis, halitosis, and candidiasis.

### BACKGROUND

Oral bacteria are highly transmissible and can trigger chronic inflammatory responses in the host that can result in tissue-destructive events such as periodontitis. Seventy-five percent of the population suffers from some form of periodontal infections or diseases. Additionally, oral bacteria have been identified as risk factors for many systemic conditions, including cardiovascular disease (CVD), diabetes, cancers, Alzheimer's disease, and others. In the case of cardiovascular conditions, these bacteria have recently been identified as causative agents. Therefore, it is important to ensure appropriate treatment.

Gram stain analysis has been used to identify oral bacterial infections and to provide a basis for selecting treatment. However, Gram strain analysis is a phenotypic analysis, in which Gram stain characterizes bacteria based on the structural characteristics of their cell walls (i.e., peptidoglycan in the cell wall). There are limitations in pinpointing causative agents at the species level and in providing precise degrees of severity of infection or microbial presence in the oral cavity.

Furthermore, the manner in which oral samples are collected from a patient's oral cavity may influence diagnosis. For example, one system currently on the market relies on paper points to test for pathogens in the subgingival region only, and does not collect saliva samples or biofilm samples from the tongue surface. Another system on the market requires a saliva sample only, which is collected from a patient who swishes and/or gargles with a solution and then expectorates into a vial. Saliva samples are indirect subgingival measurements that can detect more planktonic bacteria than biofilm samples. As a result, they are less accurate. If the patient has cleaned his or her teeth or rinsed with any mouthwash a short time prior to collection, this will alter the amount of bacteria collected in the saliva sample; therefore, this method of collection is prone to false negatives.

While systemic antibiotics have been recommended for the treatment of moderate to severe periodontal disease and halitosis caused by oral pathogens, they are not recommended for gingivitis or for controlling the exposure over time to oral pathogens that cause bacteremia without causing periodontal disease. Recent research has shown that it is important to control or treat these milder conditions.

There is a sufficient concentration of antibiotic when taken systemically to destroy some pathogens in biofilm attached to the tooth root surface and in the fluid around the root. However, the amount of antibiotic present in saliva is insufficient to destroy the planktonic form of these bacteria as well as the biofilm throughout the entire oral cavity. The use of systemic antibiotics may contribute to increased antimicrobial resistance to many antibiotics because of the low saliva concentration. Moreover, low antibiotic concentration using a systemic delivery system may be ineffective in penetrating whole mouth biofilms.

There have been some efforts to provide topical antibiotic compositions for treatment of oral infections. In one existing solution, a minocycline material in gel is applied into the periodontal pocket. While this application eliminates some bacteria; placement takes time and it does not remove any of the bacteria on the tongue and tongue base or saliva. In another solution, a doxycycline gel is applied in the gingival sulcus and the whole mouth biofilm is not treated by oral antibiotics. These are all localized treatments only. They do not treat the whole oral cavity; therefore, re-infection likely occurs.

In view of the foregoing, there exist needs for improved sampling of oral bacteria and improved methods of treating and selecting treatments for oral microbial infections.

### SUMMARY OF THE DISCLOSURE

In general, the present disclosure describes methods and kits for selecting a treatment for an oral infection in a patient.

In one implementation, there is provided a method of selecting a treatment for an oral infection in a patient. The method includes: (a) using nucleic-acid-based analysis to analyze samples of saliva, tongue biofilm, and interproximal biofilm collected from the patient's oral cavity, to determine a pathogen profile of the oral cavity; and (b) selecting an antibiotic rinse as the treatment, based on the oral infection reported in the pathogen profile.

In some aspects of the method, the oral infection is a bacterial or fungal infection. In some aspects of the method, the bacterial infection includes infection of one or more of: Aggregatibacter actinomycetemcomitans, Porphyromonas gingivalis, Treponema denticola, Tannerella forsythia, Fusobacterium nuceatum, Peptostreptococcus micros, Prevotella intermedia, Streptococcus mutans, Capnocytophaga species, Streptococcus viridans, Streptococcus pyogenes, Campylobacter rectus, Eubacterium nodatum, and Eikenella corrodens. In some aspects of the method, the fungal infection includes a Candida albicans infection.

In some aspects of the method, the oral infection includes one or more of periodontal disease, dental caries, candidiasis, or halitosis.

In some aspects of the method, the nucleic-acid-based analysis includes a polymerase chain reaction (PCR) assay. In some aspects of the method, the PCR assay includes a qPCR assay. In some aspects of the method, the qPCR assay uses absolute quantification.

In some aspects of the method, the saliva is collected under the tongue, around the mandibular teeth, and/or around the palate side of the maxillary teeth. In another aspect of the method, the tongue biofilm is collected around the tongue base and/or tongue dorsum.

In some aspects of the method, the interproximal biofilm is collected by inserting paper points into gingival sulci and/or periodontal pockets in at least 5 locations in the oral cavity. In another aspect of the method, the samples of saliva and tongue biofilm are collected using a swab.

In some aspects of the method, the pathogen profile reports counts of pre-selected target pathogens. In some aspects of the method, the counts include an absolute count of each pre-selected target pathogen.

In some aspects of the method, the pre-selected target pathogens include one or more of Aggregatibacter actinomycetemcomitans, Porphyromonas gingivalis, Treponema denticola, and Tannerella forsythia. In some aspects of the method, the pre-selected target pathogens further include Fusobacterium nuceatum and Peptostreptococcus micros. In some aspects of the method, the pre-selected target pathogens further include at least one of Prevotella intermedia, Candida albicans, Streptococcus mutans, Capnocytophaga species, Streptococcus viridans, Streptococcus pyogenes, Campylobacter rectus, Eubacterium nodatum, and Eikenella corrodens.

In some aspects of the method, the antibiotic rinse includes antibiotic particles. In another aspect of the method, the antibiotic particles include particulate metronidazole. In yet another aspect of the method, the particulate metronidazole is suspended in the antibiotic rinse. In some aspects of the method, the antibiotic particles are sized for being drawn into a gingival sulcus of the patient by a Venturi effect.

In some aspects of the method, selecting an antibiotic rinse includes evaluating the pathogen profile with at least one of the patient's habits, health history, and symptoms. In another aspect of the method, the pathogen profile reports presence of Aggregatibacter actinomycetemcomitans above a pre-determined threshold and the selected antibiotic rinse includes amoxicillin. In some aspects of the method, the selected antibiotic rinse further includes metronidazole.

In some aspects of the method, the pathogen profile reports presence of Aggregatibacter actinomycetemcomitans at or below a pre-determined threshold and the selected antibiotic rinse includes metronidazole.

In some aspects of the method, the selected antibiotic rinse further includes nystatin.

In some aspects of the method, the pre-determined threshold of Aggregatibacter actinomycetemcomitans is an absolute count of 10¹-10².

In some aspects of the method, the target pathogens include Aggregatibacter actinomycetemcomitans, Porphyromonas gingivalis, Treponema denticola, and Tannerella forsythia. In another aspect of the method, the target pathogens further include at least one of Fusobacterium nuceatum, Peptostreptococcus micros, Prevotella intermedia, Candida albicans, Streptococcus mutans, and Capnocytophaga species. In some aspects of the method, the pathogen profile reports presence of three or more of the target pathogens above their respective pre-determined thresholds and the selected antibiotic rinse includes amoxicillin.

In some aspects of the method, the pathogen profile reports presence of two or more of the target pathogens above their respective pre-determined thresholds and the selected antibiotic rinse includes amoxicillin.

In one implementation, there is provided a method for treating an oral infection in a patient. The method includes: (a) using nucleic-acid-based analysis to analyze samples of saliva, tongue biofilm, and interproximal biofilm collected from the patient's oral cavity, to determine a pathogen profile of the oral cavity; and (b) selecting an antibiotic rinse as treatment, based on the oral infection reported in the pathogen profile.

In some aspects of the method, the method further includes administering the selected antibiotic rinse as the treatment to the patient.

In one implementation, there is provided a kit for use in selecting a treatment for an oral infection in a patient. The kit includes: (a) primers specific to nucleic acid sequences of pre-selected target pathogens, for use in conducting a polymerase chain reaction (PCR) assay to analyze samples of saliva, tongue biofilm, and interproximal biofilm collected from the patient's oral cavity and determining a pathogen profile of the oral cavity; and (b) instructions to select an antibiotic rinse as the treatment based on the oral infection reported in the pathogen profile.

In some aspects of the kit, the target pathogens include one or more of Aggregatibacter actinomycetemcomitans, Porphyromonas gingivalis, Treponema denticola, Tannerella forsythia, Fusobacterium nuceatum, Peptostreptococcus micros, Candida albicans, and Streptococcus mutans. In some aspects of the kit, the target pathogens further include Prevotella intermedia and Capnocytophaga species.

In some aspects of the kit, the primers include those identified in Table 2.

In some aspects of the kit, the instructions provide thresholds of the respective target pathogens.

Additional aspects of the present disclosure will be apparent in view of the description which follows.

### BRIEF DESCRIPTION OF THE DRAWINGS

Features and advantages of embodiments of the present application will become apparent from the following detailed description and the appended drawings, in which:

FIG. 1 is a graph showing the test results of Case A of Example 2. The Y-axes represent log absolute counts of the test pathogens, and the black horizontal bars indicate normal levels (thresholds) of each of the eight target pathogens, with open bar=normal-below threshold and gray bar=infection-above threshold.

FIG. 2 is a graph showing the test results of Case B of Example 2. The Y-axes represent log absolute counts of the test pathogens, and the black horizontal bars indicate normal levels (thresholds) of each of the eight target pathogens with open bar=normal-below threshold and gray bar=infection-above threshold.

FIG. 3 is a graph showing the test results of Case C of Example 2. The Y-axes represent log absolute counts of the test pathogens, and the black horizontal bars indicate normal levels (thresholds) of each of the eight target pathogens, with open bar=normal-below threshold; hatched bar=monitor-<1 below threshold; and gray bar=infection-above threshold.

### DETAILED DESCRIPTION

The present disclosure describes methods for selecting a treatment for an oral infection in a patient. Samples are collected from multiple locations of the patient's oral cavity and include saliva, tongue biofilm, and interproximal biofilm. The samples are analyzed to determine a pathogen profile of the oral cavity, using nucleic-acid based analysis. Based on the pathogen profile, an antibiotic rinse is selected as the treatment of the oral infection.

The present disclosure also describes methods for treating an oral infection in a patient by using nucleic-acid based analysis to analyze samples from the patient's oral cavity, determining a pathogen profile, and selecting an antibiotic rinse based on the oral infection reported in the pathogen profile.

Also described herein are kits for use in selecting a treatment for an oral infection in a patient. Primers specific to nucleic acid sequences of pre-selected target pathogens are included to conduct a polymerase chain reaction (PCR) assay for analyzing samples from the patient's oral cavity and to determine a pathogen profile. The kits include instructions for selecting an antibiotic rinse as the treatment based on the oral infection reported in the pathogen profile.

Throughout this specification, numerous terms and expressions are used in accordance with their ordinary meanings. Provided below are definitions of some additional terms and expressions that are used in the description that follows.

The term “oral infection” as used herein refers to microbial overgrowth of an undesired microorganism in the oral cavity, including the enamel. The oral infection can be bacterial or fungal. Oral infections include, but are not limited to, periodontal disease, dental caries, halitosis, endodontitis, and candidiasis.

The term “biofilm” as used herein means any group of microorganisms encapsulated within a self-developed polymeric extracellular matrix. The biofilm may be adhered to a living or inert surface. For example, in the oral cavity, the biofilm may be adhered to teeth in the form of plaque.

The term “DNA analysis” or “nucleic-acid-based analysis” as used herein refers to any method of nucleic acid analysis that reveals genotype information. Genotype information may be obtained from any one of the genomic DNAs of a target organism, such as a nuclear genome, a mitochondrial genome, a chloroplast genome, or any combination thereof. Nucleic-acid-based analysis can involve the amplification of nucleic acid sequence. Examples of the types of nucleic-acid-based analysis include, but are not limited to, assays using DNA sequencing and polymerase chain reaction (PCR). In some embodiments, nucleic-acid-based analysis involves the use of reverse transcriptase (RT) PCR to identify pathogens with RNA as its genetic material.

The term “amplification” refers to the production of one or more copies of a reference nucleic acid sequence or its complement. Amplification may be linear or exponential (e.g., in a polymerase chain reaction (PCR)). A nucleic acid copy produced from amplification may not have perfect sequence complementarity or identity relative to the reference sequence. In some embodiments, the copies can include nucleotide analogs, including deoxyinosine, intentional sequence alterations (such as alterations introduced through a primer that is hybridizable, but not fully complementary, to the template), and/or sequence errors that occur during the amplification process.

The term “treatment”, “treat”, or “treating” refers to clinical intervention in an attempt to alter the natural course of the individual being treated, and can be performed either for prophylaxis or during the course of clinical pathology. Effects of treatment can include preventing occurrence or recurrence of disease, alleviating symptoms, diminishing any direct or indirect pathological consequences of disease, decreasing the rate of disease progression, ameliorating the disease state, minimizing the clinical impairment or symptoms resulting from the disease, diminishing any pain or discomfort suffered by the subject, remission, or improved prognosis. As used herein, “treatment” includes therapy.

The term “pathogen” or “oral pathogen” as used herein refers to bacteria and fungi which are resident in the oral cavity and have been implicated in various disease or infectious states, both topically (in the oral cavity) or systemically (from inside the body). Exemplary disease or infectious states include, without limitation, periodontitis, gingivitis, caries, endodontitis, thrush, cardiovascular disease (CVD), pediatric autoimmune neuropsychiatric disorders associated with streptococcal infections (PANDAS), diabetes, respiratory disease, hypertension, kidney disease, rheumatoid arthritis, pregnancy complications, and a variety of cancers. Oral pathogens include, but are not limited to, Aggregatibacter actinomycetemcomitans, Porphyromonas gingivalis, Treponema denticola, and Tannerella forsythia (also known as Bacteroides forsythus or Tannerella forsythensis), Fusobacterium nuceatum, Peptostreptococcus micros (also known as Parvimonas micra), Prevotella intermedia, Streptococcus mutans, Streptococcus viridans (also known as viridans streptococcal species), Streptococcus pyogenes (also known as Group A streptococcus β-hemolytic), Campylobacter rectus, Eubacterium nodatum, Eikenella corrodens, Capnocytophaga species, and Candida albicans.

The term “pathogen profile” as used herein refers to the presence of bacterial or fungal pathogens that cause or contribute to oral infection, inflammation, and/or systemic infections/inflammation, for which pathogen-specific nucleic acids can be detected by nucleic-acid-based analysis, including information or data associated thereto.

The term “periodontal disease” as used herein encompasses a number of diseases that affect the supporting tissues of the teeth and includes, for example, gingivitis and periodontitis.

The term “dental caries” as used herein refers to conditions also known as tooth decay, cavities, or caries, which is a breakdown of teeth due to activities of bacteria. The cavities may be a number of different colors from yellow to black.

The term “endodontitis” or “endodontic infection” as used herein refers to infection of the dental root canal system; it is considered the major cause of apical periodontitis.

The term “halitosis” as used herein means oral malodor or bad breath, especially in association with resident oral bacteria.

The term “candidiasis” as used herein refers to fungal conditions caused by fungi of the genus Candida, including those known as oral candidiasis, yeast infections, or thrush.

The term “antibiotic rinse” as used herein refers to a liquid formulation containing at least one antibiotic for swishing and gargling in an oral cavity. The antibiotic rinse can be a solution, suspension, or colloid. In the ordinary course of usage, such a rinse is not intentionally swallowed for purposes of systemic administration of particular therapeutic agent(s), but, rather, is retained in the oral cavity for a time sufficient to contact substantially all of the dental surfaces and/or oral tissues in the oral cavity.

The term “primer” includes single-stranded polynucleotide that is capable of hybridizing to nucleic acid and allowing the polymerization of a complementary nucleic acid, generally by providing a free 3′-OH group.

The term “sequencing” and its variants include obtaining sequence information from a strand of a nucleic acid molecule, typically by determining the identity of at least some nucleotides (including their nucleobase components) within the nucleic acid molecule. The term sequencing may also refer to determining the order of nucleotides (base sequences) in a nucleic acid sample (e.g. DNA or RNA). Many techniques are available and known to a person skilled in the art, such as Sanger sequencing, high-throughput sequencing technologies (such as the GS FLX platform offered by Roche Applied Science, Penzberg, Germany, based on pyro sequencing), and the like. High-throughput sequencing technologies refer to sequencing technologies having increased throughput as compared to traditional Sanger- and capillary-electrophoresis-based approaches (e.g., with the ability to generate hundreds of thousands or millions of relatively small sequence reads at a time). These high-through-put sequencing technologies include, but are not limited to, sequencing by synthesis, sequencing by ligation, pyrosequencing, sequencing by hybridization, polony sequencing, and/or the like.

Unlike Gram staining, nucleic-acid-based analysis is genotypic rather than phenotypic, and can provide a more accurate profile of the microbial population in a given sample. Nucleic-acid-based analysis includes nucleic-acid-based amplification methods, such as polymerase chain reaction (PCR), and high-throughput sequencing technologies.

NGS encompasses non-Sanger-based high-throughput DNA sequencing technologies that make it possible to sequence DNA and RNA much more quickly and cheaply when compared to Sanger sequencing. Recent studies have suggested the possibility of using NGS platforms for both quantification of the relative abundance of microbial genes in a sample and absolute quantification through the use of internal standards.

A number of molecular tests, including qPCR and digital PCR, are known to be highly quantitative; however, they are also generally regarded as being qualitative in that they provide results in the form of absolute gene copy number and profiles of species composition generated from amplicon sequencing.

In one embodiment, a PCR assay is used to identify and characterize oral pathogens. In some embodiments, the PCR assay is a real-time PCR or quantitative PCR (qPCR) assay.

When the results of qPCR are calculated, either absolute or relative quantification can be used. In one embodiment, absolute quantification uses the standard curve method in which unknowns are quantitated based on a known quantity. First, a standard curve needs to be prepared and then unknowns can be compared to the standard curve and a value can be extrapolated. In another embodiment, relative quantification is used to analyze changes in gene expression in a given sample by analyzing the gene expression relative to another reference sample, such as an untreated control sample. In some embodiments, when analyzed by qPCR, the oral pathogens in the sample are quantitated by absolute quantification.

In certain embodiments, the methods of detecting and identifying microorganisms described herein rely on pre-selection of oral pathogens that have been identified for nucleic-acid-based analysis. In some embodiments, the target pathogens can be selected from the oral pathogens that have been known to be infectious agents of periodontal disease.

As discussed by Kim and Amar (“Periodontal disease and systemic conditions: a bidirectional relationship”, Odontology, 2006 September, 94(1): pp. 10-21), the most frequently identified periodontal pathogens include three microaerophilic species (Actinobacillus actinomycetemcomitans, Campylobacter rectus, and Eikenella corrodens) and seven anaerobic species (Porphyromonas gingivalis, Bacteroides forsythus, Treponema denticola, Prevotella intermedia, Fusobacterium nucleatum, Eubacterium, and spirochetes). The pathogens can be divided into two main clusters of microorganisms, namely the “red” and “orange” complexes. “Green”, “yellow”, and “purple” complexes are defined as the bacterial colonies that form on the tooth surface prior to the colonization of the “orange” and “red” complexes.

The “red” complex consists of three tightly related species: T. forsythensis, P. gingivalis, and T denticola. This complex is strongly related to pocket depth and bleeding on probing. The “range complex” includes F. nucleatum/periodonticum subspecies, P. intermedia, P. nigrescens, Peptostreptococcus micros, C. rectus, C. gracilis, C. showae, Eubacterium nodatum, and Streptococcus constellatus, and seems to precede colonization by species of the “red” complex. The “yellow” complex comprises six Streptococcus species—Streptococcus sp., S. sanguis, S. oxalis, S. intermedius, S. gordonii, and S. mitis—while Capnocytophaga ochracea, Capnocytophaga gingivalis, Capnocytophaga sputigena, E. corrodens, and A. actinomycetemcomitans serotype a make up the “green” complex. The “purple” complex consists of Veillonella parvula, Actinomyces odontolyticus, A. actinomycetemcomitans serotype b, Selenomonas noxia, and Actinomyces naeslundii genospecies 2 (Actinomyces viscosus), but these do not constitute any cluster or ordination group. Various herpes viruses, such as human cytomegalovirus (HCMV) and Epstein-Barr virus (EBV-1), are also known as pathogens implicated in destructive periodontal disease.

The oral-systemic link can be considered in pre-selecting target pathogens since the associations between periodontal disease and systemic diseases are widely accepted. Periodontal disease has been shown to result in inflammation in parts of the body beyond the oral cavity. This inflammation and the causative periodontal pathogens have been implicated as contributing factors, through a variety of pathways, in a multitude of systemic diseases and conditions, such as cardiovascular disease (CVD), pediatric autoimmune neuropsychiatric disorders associated with streptococcal infections (PANDAS), diabetes, respiratory disease, hypertension, kidney disease, rheumatoid arthritis, pregnancy complications, and a variety of cancers. PANDAS is, in part, caused by an autoimmune response to a strep infection. Its symptoms mimic those of obsessive compulsive disorder (OCD) and attention deficit hyperactivity disorder (ADHD), and include motor and verbal tics, and treatment involves medication and cognitive behavioral therapy.

In some embodiments, the pre-selected target pathogens include those that cause periodontal disease, halitosis, tooth decay, oral infections, systemic infections, and inflammation/inflammatory products that relate to oral and systemic diseases, such as PANDAS, CVD, and the like.

Table 1 illustrates a set of specific target pathogens according to one embodiment. As shown in Table 1, those pathogens may be categorized into several groups. The first group of the target microorganisms are the most harmful periodontal pathogens that include Aggregatibacter actinomycetemcomitans, Porphyromonas gingivalis, Treponema denticola, and Tannerella forsythia. The next group of selected harmful periopathogens include Fusobacterium nuceatum, Peptostreptococcus micros, and Candida albicans. In some embodiments, as a high-risk cariogenic pathogen, Streptococcus mutans can also be selected. In some embodiments, additional target pathogens can be selected in order to generate a more complete microbial profile of a patient's oral cavity. For example, in some embodiments, Prevotella intermedia can be added as a member of the moderate-risk periopathogen group. In further embodiments, Capnocytophaga species can be added as members of the low-risk periopathogen group.

TABLE 1
Exemplary Selection of Target Pathogens

| Risk | Pathogen | |
|---|---|---|
| Classification | Classification | Target Pathogenic Species or Genus |
| High-Risk | | Aggregatibacter |
| Periopathogens | | actinomycetemcomitans (Aa) |
| | Red Complex | Porphyromonas gingivalis (Pg) |
| | Bacteria | Treponema denticola (Td) |
| | | Tannerella forsythia (Tf) |
| Moderate-Risk | Orange Complex | Peptostreptococcus micros (Pm) |
| Periopathogens | Bacteria | Fusobacterium nucleatum (Fn) |
| | | Prevotella intermedia (Pi) |
| | Yeast | Candida albicans (Ca) |
| Low-Risk | | Capnocytophaga species (Cs) |
| Periopathogens | | |
| High-Risk | | Streptococcus mutans (Sm) |
| Cariogenic | | |
| Pathogen | | |

In some embodiments, the list of target pathogens may also include Streptococcus viridans or Streptococcus pyogenes. In some embodiments, the list of target pathogens may further include, as members of the moderate-risk periopathogen group, Campylobacter rectus, Eubacterium nodatum, or Eikenella corrodens.

Table 2 shows the exemplary selection of target pathogens of Table 1 and the relevant primers for DNA analysis (e.g., qPCR) according to one embodiment.

TABLE 2
Exemplary Selection of Target Pathogens and
Relevant Primers

| Target | |
|---|---|
| Pathogen | Primer Sequence 5′ to 3′ |
| Td | F-primer: CCTTGAACAAAAACCGGAAA |
| | R-primer: GGGAAAAGCAGGAAGCATAA |
| Aa | F-primer: CTTACCTACTCTTGACATCCGAA |
| | R-primer: ATGCAGCACCTGTCTCAAAGC |
| Pg | F-primer: TGGTTTCATGCAGCTTCTTT |
| | R-primer: TCGGCACCTTCGTAATTCTT |
| Tf | F-primer: GATAGGCTTAACACATGCAAGTC |
| | R-primer: GTTGCGGGCAGGTTACATAC |
| Fn sp. | F-primer: AAGCGCGTCTAGGTGGTTATGT |
| | R-primer: TGTAGTTCCGCTTACCTCTCCAG |
| Pm | F-primer: AAACGACGATTAATACCACATGAGAC |
| | R-primer: ACTGCTGCCTCCCGTAGGA |
| Pi | F-primer: TCCACCGATGAATCTTTGGTC |
| | R-primer: ATCCAACCTTCCCTCCACTC |
| Ca | F-primer: GCCGGTGACGACGCTCCAAGAGCTG |
| | R-primer: CCGTGTTCAATTGGGTATCTCAAGGTG |
| Sm | F-primer: GCCTACAGCTCAGAGATGCTATTCT |
| | R-primer: GCCATACACCACTCATGAATTGA |
| Cs | F-primer: AAGTCGAGGGAGAAGCCCT |
| | R-primer: TCCAAATTTCTTCGGGGCTATC |
| | Universal Primers |
| | F-primer: TCCTACGGGAGGCAGCAGT |
| | R-primer: GGACTACCAGGGTATCTAATCCTGTT |

In the embodiment shown in Table 1, universal primers are used to assess total bacterial load in a sample by amplifying conserved regions in the genetic material of bacteria, such as the 16S rDNA region.

Once target pathogens are selected or pre-selected, thresholds of “acceptable risk” or “normal healthy ranges” can be defined for the chosen pathogens before incorporating quantification of pathogens by nucleic-acid-based approaches into the methods of identifying appropriate treatment.

In some embodiments, the threshold levels of, for example, eight oral pathogens (Table 3) are established by using the absolute count method on clinical specimens. Such thresholds are described “Subgingival periodontal pathogens associated with chronic periodontitis in Yemenis”, Al-hebshi et al., BMC Oral Health, 2014, 14:13; and “Detection of Candida in Concentrated Oral Rinse Cultures by Real-Time PCR”, White et al., Journal of Clinical Microbiology, May 2004, pp. 2101-2107, both of which are incorporated herein by reference in their entireties. In some embodiments, the thresholds are based on current literature and are reported in log DNA copies per sample.

TABLE 3
Thresholds of Eight Test Species Pathogens per Sample (in log
absolute count, along with absolute count in parentheses)

| | | |
|---|---|---|
| Td | Treponema denticola | 5 (10⁴-10⁵) |
| Aa | Aggregatibacter actinomycetemcomitans | 2 (10¹-10²) |
| Pg | Porphyromonas gingivalis | 4 (10³-10⁴) |
| Tf | Tannerella forsythia | 5 (10⁴-10⁵) |
| Fn | Fusobacterium nucleatum | 6 (10⁵-10⁶) |
| Pm | Peptostreptococcus micros | 4 (10³-10⁴) |
| Ca | Candida albicans | 3 (10²-10³) |
| Sm | Streptococcus mutans | 6 (10⁵-10⁶) |

In some embodiments, the thresholds of pathogens which are not listed in Table 3 can be determined by a person skilled in the art in accordance with the present disclosure and in view of Al-hebshi et al., supra, and White et al., supra.

The selection of the target pathogens and determination of their thresholds are followed by amplification of a nucleic acid sequence specific to each target pathogen. The amplified nucleic acid sequence is detected and the presence of the amplified nucleic acid sequence indicates the presence of the specific target pathogen in a given sample.

In various embodiments, the present methods are used to determine the type and number of each detected pathogen from the patient's oral cavity, assess the risk based on the results of the assay, and select an antibiotic and/or an antifungal in a particulate rinse/cream form to predictably treat periodontal disease, dental caries, candidiasis, halitosis, CVD, PANDAS, and the like.

As discussed above, the present methods can be used to identify some of the most harmful oral bacteria that have been known to cause periodontal diseases, halitosis, tooth decay, and the like. In order to treat these infections, the causative agents are detected and quantitated. Once this is accomplished, then antibiotics are selected to kill the bacterial and/or fungal pathogens causing the infection. In some embodiments, antibiotics known to be especially effective against a certain pathogen are selected. Finally, effective maintenance rinse protocols targeting the pathogens or causative agents are put in place to prevent a relapse of the infection.

Since the selection of the appropriate antibiotics for treating the oral infections relies on the precise detection and quantification of the causative oral pathogens, whole mouth sampling, which includes collection of both biofilm and planktonic bacteria/fungi, is used. Either salivary sample or subgingival sample alone is insufficient. Taking samples of biofilm above and below the gum line (including the tongue biofilm and the interproximal biofilm) as well as from the whole mouth, including saliva, can provide more comprehensive results.

In some embodiments, in order to perform the sampling of the entire oral cavity, including tongue, saliva, throat, sub and supra gingival, a long handled swab can be used to collect tongue biofilm and saliva around the teeth, followed by the use of five paper points placed into selected areas between the teeth and into the periodontal pocket. For example, samples can be collected from the tongue base, tongue dorsum, lingual and palatal gingival tissue, and saliva for a whole mouth planktonic and biofilm sample using the long handled swab. The swab can then be separated from the handle and placed into a collection tube. In some embodiments, paper points can be used to collect samples from the gingival sulcus and periodontal pockets (including the interproximal biofilm); these can also be placed in the collection tube for a nucleic-acid-based analysis.

When a sample is analyzed using a qPCR absolute method, the absolute count (e.g., target DNA copies per sample) for each selected pathogen is provided by using a standard curve or calibration curve. The resulting report then recommends the basis for treatment of the oral infections using specific antibiotic rinses (and creams).

If the practitioner further requires the location, type, and density of bacteria, a Gram stain analysis can be used. Samples can be taken from 6 specific sites of the patient's oral cavity—tongue base, tongue dorsum, upper right, upper left, lower left and lower right quadrants, and placed on to a slide and sent to a lab for analysis. The pathogen groups that are analyzed are Gram positive bacilli and cocci; Gram negative straight and curved bacilli and cocci; fusiforms; spirochetes; yeast; amoebae; and neutrophils.

Rinses are more effective in delivering antibiotics for oral infections than systemic administration or applications of creams or gels. When compared to systemic antibiotics offered by some existing systems, antibiotics in rinse form can offer undiluted antibiotic in extremely high concentration with less bacterial resistance and fewer systemic side effects. In addition, a rinse form of antibiotic can be more effective than a cream or gel form because the antibiotic can be applied to the entire oral cavity (throat, tongue, gums, tonsils, and teeth) by swishing and gargling, thereby reducing any potential re-infection.

In some embodiments, antibiotic rinse formulations may contain metronidazole in particles suspended in water. Metronidazole can be the major base ingredient of the antibiotic rinse formulations because it destroys a large number of microorganisms including Entamoebae gingivalis. Entamoebae gingivalis is found in the mouth inside the gingival pocket biofilm near the base of the teeth and in periodontal pockets, and found in 95% of people with gum disease and rarely in people with healthy gums.

In principle, selection of the antibiotic rinses is based on the types and amount of bacteria that are reported in the nucleic-acid-based analysis. In one embodiment, if the report contains Aggregatibacter actinomycetemcomitans over its threshold level, then an antibiotic rinse comprising metronidazole and a small amount of amoxicillin may be selected. Aggregatibacter actinomycetemcomitans responds best to amoxicillin and about 50% of the time to metronidazole. If Aggregatibacter actinomycetemcomitans is present at threshold or below, then the selected antibiotic rinse may comprise metronidazole without amoxicillin.

In some embodiments, antibiotic rinses may include an antifungal agent to control opportunist yeasts. When an antifungal agent is included in an antibiotic rinse, it can prevent oral thrush resulting from systemic antibiotic use. Exemplary antifungal agents include nystatin, which can be an essential ingredient of the rinse formulations when Candida is detected as a component of oral biofilms. Recent studies have found that Candida may exacerbate the amount of acid production when combined with Streptococci.

The combination of metronidazole and nystatin can be used to control most of the microorganisms including Candida. In general, amoxicillin can be used if the bacterial load of Aggregatibacter actinomycetemcomitans in a sample is greater than the pre-determined threshold thereof. In one embodiment, while metronidazole can be used to control the other selected pathogens; if the density of pathogens is high (i.e., many of these pathogens are identified above their threshold levels), the combination of metronidazole, nystatin, and amoxicillin may be used. In some embodiments, antibiotic resistance profiles for specific pathogens can be taken into consideration in determining appropriate treatment. For example, in one embodiment, Eubacterium nodatum and Eikenella corrodens are resistant to metronidazole but responsive to amoxicillin; therefore, the proper treatment may include amoxicillin.

Several formulations of these antibiotic rinses are available: metronidazole/nystatin formulation for children under 12 years (FM2); metronidazole/nystatin formulation for adults (FM4); metronidazole/amoxicillin/nystatin for an individual who has a heavy microbial load and/or where nucleic-acid-based analysis shows the presence of bacteria which do not respond effectively to metronidazole alone (FM4-Amox); and metronidazole/nystatin in a cream form (FM3) that is placed in localized areas of an oral cavity.

Customized rinses may also be formulated to treat specific conditions such as PANDAS, strep throat, and tonsillitis. The FM3 antibiotic cream has the advantage of being easy to use for the dental practitioner as well as the patient. It can be delivered in a syringe to the area or applied by the patient using Soft-Picks™ or other interdental aids coated with the antibiotic cream.

Antibiotic rinses can be more effective with antibiotic particles suspended in the rinse liquid. In some embodiments, topical antibiotic antifungal rinses for the present methods can be colloidal suspensions of antibiotic particles. The rinses contain less than half the systemic dose of the antibiotic; however, 3,000 to 4,000 times the concentration of the same oral dose can be present in saliva because the medication is directly applied to the site of infection without dilution, absorption, or metabolism issues.

In some embodiments, patients use these rinses for two weeks, three times a day (e.g., 2-5 ml per rinse, preferably 3 ml per rinse), swishing, gargling vigorously for 30 seconds, and then spitting out the medication. In that time, there is sufficient exposure to the antibiotic for particles to coat the tongue and tongue base and to enter into the gingival (crevicular) sulcus. After each rinsing session, the patient may be advised to use Soft-Picks or other interdental aids coated with the antibiotic rinse to clean in between the teeth. This routine can place more particles into the sulcus and result in greater reduction of subgingival biofilm and subsequently better healing.

In some embodiments, vigorous rinsing, along with flossing to move particles into the sulcus, can be aided by the phenomenon called the Venturi effect. The crevicular pressure drops when the particles spin over periodontal pockets and this drop in pressure drags the particles down to the base of the pockets. The antibiotic particles may reach the base of 6-7 mm pockets while fluids, in contrast, May only penetrate 1 mm. The concentrated antibiotic particles can then act on the biofilm found at the base of these pockets. The antibiotic particles remain in the sulcus and attached to oral biofilms, slowly dissolving and releasing antibiotic over the 8 hours until the patient rinses again.

When antibiotic rinses are used, the amount of antibiotic present in the periodontal pocket is undiluted since it is in originally-formulated form; it is unaffected by absorption issues and not broken down by liver enzymes. Over a period of two weeks, considerable healing takes place with, for example, a decrease of 87% in periodontal bleeding points and a considerable shift (about 2+ mm) in pocket depth.

In some embodiments, antibiotic rinses can be prepared in the manners as described in Canadian Patent Application No. 2,458,219 A1, published on Mar. 6, 2003, and in “Treatment of oral malodor and periodontal disease using an antibiotic rinse”, Southward and Bosy, General Dentistry, July 2013, pp. 41-45. Both references are incorporated herein by reference in their entireties.

In some embodiments, the rinse formulations may contain metronidazole at a concentration of about 5 to about 200 mg/ml, preferably of about 20 to about 50 mg/ml. In some embodiments, these formulations may further contain nystatin at a concentration of about 20,000 to about 600,000 IU/ml (based on 100,000 IU per gram, which equates to a range of about 200 to about 6000 mg/ml), preferably of about 100,000 IU/ml.

The rinse formulations, made up of antibiotic particles (metronidazole) suspended in an antifungal solution (nystatin), travel into the sulcus and are effective in decreasing bleeding and pocket depth. The formulations may optionally include additives such as a corticosteroid, a pain relief agent, a self-sterilizing agent, a flavouring agent, or a colouring agent as appropriate.

In some embodiments, treatment recommendations and selection of antibiotic rinses may depend on the following factors: description of patient habits (e.g., smoking, alcohol intake, diet, and the like), relevant health history, periodontal disease, caries, halitosis, description of symptoms, type of microorganisms present, and density of microorganisms. The treatment recommendations can include a follow-up maintenance regimen.

A reliable maintenance rinse protocol can help a patient to prevent or delay recurrence of oral infection. In one embodiment, the two weeks of treatment is followed by another two weeks of antimicrobial rinsing, twice a day, using a 0.2% chlorhexidine (about 4 ml per rinse). Soft-Picks or other interdental aids wet with the chlorhexidine rinse can be used to clean in between the teeth. Chlorhexidine is effective in decreasing both Streptococci, such as S. mutans, and other gram-positive pathogens that may have increased during the treatment with antibiotic preparations.

In some embodiments, if there is a lot of bleeding, deeper pockets, or odours between the teeth, then the patient may be required to use Soft-Picks coated with antibiotic cream (FM3), which is composed of metronidazole particles, nystatin, and flavouring in a cream base such as Glaxal Base™ or Eucerin™. This medication can be easily applied by the patient or by the practitioner and is used to treat failing implants, resistant pockets of bacteria, and denture stomatitis, and as post-surgical treatment. It can be used twice a day for one week and can be repeated if required. The application of such a cream can reduce pathogens.

Upon the completion of the 4 weeks of rinsing (two weeks antibiotic rinse and two weeks chlorhexidine rinse), the patient may return to the dental professional for evaluation. At evaluation, the amount of healing can be assessed. If the tissues are healthy, then the patient can start a regimen of good oral care and a maintenance rinse. However, if there are areas of bleeding, pockets, or odour that still remain, the patient may use the maintenance system for two weeks and then repeat the application of antibiotic cream FM3 twice a day for two weeks. The patient may first rinse with his/her maintenance rinse and then clean in between all the teeth using Soft-Picks or other interdental aids coated with the antibiotic cream. Special attention may be given to those areas that have not responded.

It is important at that appointment for the dental professional to check on the patient's oral hygiene care. This can be accomplished using a two-tone disclosing solution that identifies any remaining biofilm and clearly shows the patient what areas are missed during daily cleaning. This is important for identifying corrective measures to be put in place to ensure that the infection may return over time. Therefore, at this time, the patient can demonstrate his/her daily technique and corrections can be made to both the overall cleaning and the cleaning between the teeth (interdental cleaning) for maximum removal of biofilm.

In some embodiments, a daily maintenance mouthwash can be introduced based on the patient's needs. This mouthwash can be used twice a day, in the morning after brushing and in the evening. The goal of this mouthwash is to decrease the planktonic or free floating bacteria as well as those in the biofilm on the visible portion of the teeth. This mouthwash is usually bacteriostatic (OraVital CDLx™) but can be bacteriocidal (OraVital CPCx™).

At the end of two weeks, the patient may resume the maintenance routine, including the mouth rinse, for a period of 4 weeks. At the end of the 4-week period, the patient may again use the FM3, first rinsing with the maintenance rinse, and then applying the antibiotic cream with Soft-Picks or other interdental aids to all the teeth. This may be repeated twice a day for one week. Following the week, the patient may return to the maintenance routine and also be evaluated for progress.

It is recommended that the antibiotic rinses and creams be prescribed once every six months (or longer) on an as-need basis. If there is sufficient healing, then the patient can continue to use the maintenance program.

The present methods are described in the following examples, which are set forth to aid in the understanding of the invention, and should not be construed to limit in any way the scope of the invention as defined in the claims which follow thereafter.

### EXAMPLES

### Example 1—DNA Analysis

a. Pre-Selection of Target Pathogens

The following eight oral pathogens were selected as targets for DNA analysis of oral samples:

### Periodontal Risk Pathogens:

Porphyromonas gingivalis [Pg]—A prominent player in progressive periodontal disease, known to invade and destroy the tissues supporting the tooth that may lead to tooth loss.
Treponema denticola [Td]—An oral spirochete, strongly associated with poly-microbial periodontal infections and chronic periodontal disease progression. Its motility plays a pivotal role in tissue invasion and proteolytic activity in tissue destruction and host immunosuppression.
Tannerella forsythia [Tf]—A pathogen strongly associated with the pathogenesis and progression of destructive forms of periodontitis, particularly advanced and recurrent periodontitis. Known to adhere to host cells, invade tissues, and contribute to host immunosuppression.
Aggregatibacter actinomycetemcomitans [Aa]—An aggressive bacterium associated with juvenile periodontitis and advanced adult periodontal disease. This pathogen has the ability to stimulate cytokines that are capable of stimulating bone destruction.
Fusobacterium nucleatum [Fn]—This bacterium has been recognized as part of the subgingival microbiota, prevalent in patients with periodontal disease. It also produces large amounts of volatile sulfur compounds (VSCs), which are major substances associated with halitosis. F. nucleatum can aggregate with other bacteria to form plaque biofilms on the tongue that can increase oral malodors and progress to chronic halitosis.
Peptostreptococcus micros [Pm]—A recognized pathogen associated with progressive periodontal disease and also endodontic abscesses.

### Caries Risk Pathogens:

Streptococcus mutans [Sm]—This is one of the key etiologic agents involved in the onset and progression of carious lesions resulting in rampant destruction of the smooth surfaces of the teeth.

### Yeast:

Candida albicans [Ca]—An opportunistic fungus, commonly found in the oral cavity. Ca often participates in the formation of poly-microbial biofilms on soft tissues and acrylic surfaces. It is frequently detected along with periodontal and cariogenic pathogens in a bacterium-fungus symbiotic relationship that enhances the transition from a healthy to a disease state.
b. Sampling from Oral Cavity of Patients

Saliva and tongue biofilms from the oral cavity of each patient were collected using a swab. Biofilms in between the teeth (i.e., interproximal biofilms) were collected using five paper points.

The long-handled and sterile buccal swab was applied to the tongue base and tongue dorsum to collect the biofilms and saliva. The swab was then moved under the tongue and around the mandibular teeth, then around the palate side of the maxillary teeth to collect samples from lingual gingival tissue and saliva. The swab was then separated from the handle and placed into a collection tube containing 500 microlitres of phosphate saline buffer (PBS).

Five sterile paper points (#45 Dia, Henry Schein) were used, one in each quadrant, into the deepest pocket. The paper points were inserted into the gingival sulcus and periodontal pockets between the teeth into the space around the tooth. They were left for approximately 15 seconds to collect the crevicular fluid containing bacteria, and then removed. Once removed, the paper points were also placed in the collection tube along with the swab.

The collection tube with the samples was closed and stored at 4 degrees until sent to the Biofilm Diagnostics Lab for DNA extraction and analysis.

c. DNA Extraction from Oral Samples

The contents of the collection tube were prepared for qPCR analysis by first extracting genomic DNA using MO BIO™ DNA Isolation Kit (MO BIO, Carlsbad, Calif., USA) as recommended by the supplier. Briefly, samples were added to a bead beating tube for rapid cell lysis using both mechanical and chemical methods. Total genomic DNA was captured on a silica membrane in columns and then washed and eluted from the membrane. DNA extracts were then quantified by spectrum photometer reading (NanoDrop, Wilmington, Del., USA) and were subsequently ready for PCR analysis.

d. Quantitative Real-Time PCR Assays

Twenty nanograms of DNA extract from oral biofilm samples was amplified using qPCR with species specific primer pairs for A. actinomycetemcomitans, T denticola, P. gingivalis, T. forsythia, F. nucleatum, P. micros, C. albicans, and S. mutans were detected and quantified in the DNA extracts using SYBR Green™ real-time PCR technology.

Each reaction was run in duplicate using the StepOnePlus™ qPCR system (Applied Biosystems, Foster City, Calif., USA). All assays were performed, including nontemplate control to exclude reagent contamination. Plasmids containing synthetic DNA target sequences (GeneArt Synthesis, California, USA) for 8 oral species associated with periodontal disease, caries and candidiasis were used as standard for the absolute quantification method (Table 4).

TABLE 4
Sequences of Primers used in the Quantitative PCR Assays

| Test | | Target |
|---|---|---|
| Species | Primer Sequence 5′ to 3′ | gene |
| Td | F-primer: CCTTGAACAAAAACCGGAAA | waaG |
| | R-primer: GGGAAAAGCAGGAAGCATAA | |
| Aa | F-primer: CTTACCTACTCTTGACATCCGAA | 16S rRNA |
| | R-primer: ATGCAGCACCTGTCTCAAAGC | |
| Pg | F-primer: TGGTTTCATGCAGCTTCTTT | waaA |
| | R-primer: TCGGCACCTTCGTAATTCTT | |
| Tf | F-primer: GATAGGCTTAACACATGCAAGTC | 16S rRNA |
| | R-primer: GTTGCGGGCAGGTTACATAC | |
| Fn sp. | F-primer: AAGCGCGTCTAGGTGGTTATGT | 16S r RNA |
| | R-primer: TGTAGTTCCGCTTACCTCTCCAG | |
| Pm | F-primer: AAACGACGATTAATACCACATGAGAC | 16S rRNA |
| | R-primer: ACTGCTGCCTCCCGTAGGA | |
| Ca | F-primer: GCCGGTGACGACGCTCCAAGAGCTG | 18S rRNA |
| | R-primer: CCGTGTTCAATTGGGTATCTCAAGGTG | |
| Sm | F-primer: GCCTACAGCTCAGAGATGCTATTCT | gftB |
| | R-primer: GCCATACACCACTCATGAATTGA | |
| | Universal primers | |
| | F-primer: TCCTACGGGAGGCAGCAGT | |
| | R-primer: GGACTACCAGGGTATCTAATCCTGTT | |

The PCR reactions were carried out under the following conditions: initial enzyme activation at 95° C. for 2 minutes followed by 40 cycles of denaturation at 95° C. for 15 seconds, and annealing/extension at 60° C. for 1 minute. Reading (absolute count in copies/reaction) was interpreted against the standard curve for each pathogen obtained from the amplification of related plasmid DNA. Threshold levels were based on the current literature and were reported in log copies per sample.

### Example 2—Whole-Mouth Microbial Profiling

The results of the qPCR assays of Example 1 were evaluated, and treatment recommendations were provided accordingly.

a. Case A

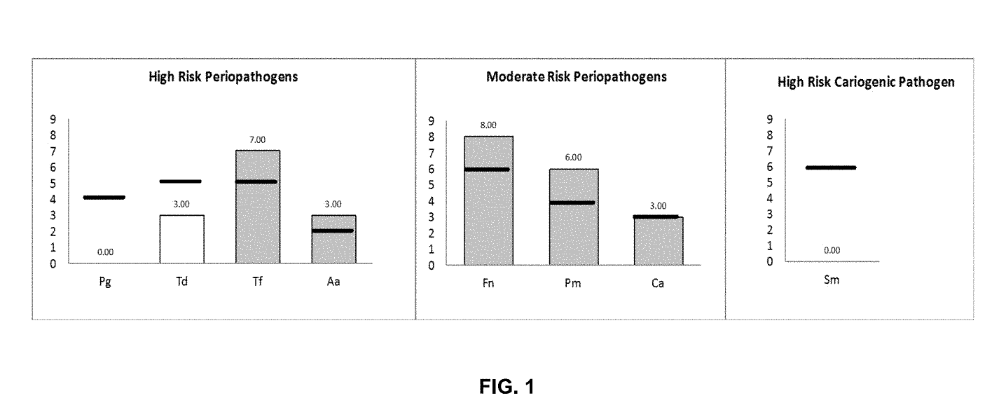

The patient information and test results are provided in Table 5 and FIG. 1.

TABLE 5
Patient and Test Information

| Patient Information | Test Information |
|---|---|
| Name of Patient: n/a (Patient A) | Ordered By: n/a |
| Date of Birth: n/a | Clinic Name: n/a |
| Gender: male | Address: n/a |
| Medical/Dental concerns: bad taste | State/Province/Country: n/a |
| Maximum Pocket Depth: 5 mm | Postal or Zip Code: n/a |
| Periodontal disease: moderate | Date Sample Collected: |
| Allergies: not reported | Date Sample Received: |
| Antibiotic history: used in the last 3 months | Report Date: |

FIG. 1 illustrates the levels of target pathogens from the sample of Patient A. In summary, high and moderate risk pathogen were detected for Patient A. The detailed report was as follows:
Periodontal risk: The high-risk periodontal pathogen P. gingivalis was not present. T. denticola was at the monitor level, but T. forsythia and A. actinomycetemcomitans were still found above threshold level. Moderate-risk pathogens F. nucleatum and P. micros were also found above threshold. All periopathogens detected here and other bacteria not included in this test may contribute to the still reported bad taste and moderate periodontal disease.
Caries risk: S. mutans was not detected in the sample.
Candidiasis: C. albicans was found elevated.

Treatment was selected and recommended based on medical history, periodontal charting, and the above report. In this Example, the specific treatment recommendations for Patient A were as follows:
1. There is a significant number of oral pathogens present in this report. Therefore, Patient A may benefit from using the antibiotic rinse FM4+Amox. The patient should use this rinse 3 times a day until completed, rinsing vigorously for 20 seconds, gargling for 10 seconds*, and then spitting the mixture out. Fusobacterium nucleatum, Peptostreptococcus micros, and Tannerella forsythia are capable of volatile sulfide compound production and will contribute to Patient A's bad taste concerns. The metronidazole in the rinse is effective against these bacteria and the amoxicillin will help to decrease both Aa and Tf. The rinse preparation also contains nystatin to decrease the levels of Candida.
2. *If the halitosis includes a bad taste from the back of the tongue, after swishing vigorously, a 10 second gargle with the antibiotic rinse is recommended prior to spitting out.
3. Tongue cleaning is recommended prior to rinsing to remove the coating and to allow for closer contact with the antibiotic rinse.
4. Patient A should use Soft-Picks coated with FM4+Amox rinse to clean between the teeth. This is an important step that will help break up the biofilm and also move particles into the sulcus. The patient should pay special attention to those areas with odour.
5. After completing the antibiotic rinse, Patient A should rinse with Cx620™ or Cx220™ (0.2% chlorhexidine gluconate) twice a day for two weeks and continue to use Soft-Picks twice a day to clean between the teeth, but this time wet with the chlorhexidine. The Soft-Picks will clean interdentally and also release some chlorhexidine into the sulcus. This will continue to decrease the biofilm and result in odour reduction or elimination. Chlorhexidine is also very effective in the reduction of Candida albicans.
6. At the revaluation appointment, use disclosing solution to identify biofilm, especially at the gingival margin and in between the teeth as this will help Patient A to focus on those areas when brushing.
7. Maintenance should consist of effective interdental cleaning and a daily mouthwash such as OraVital CDLx (ingredients: sodium chlorite, chlorine dioxide, and 3.1% xylitol) or SmartMouth™ (ingredients: sodium chlorite and zinc chloride).
8. Soft-picks that are wet with the maintenance rinse should be used several times a week to continue controlling interproximal odor causing biofilm.
   b. Case B

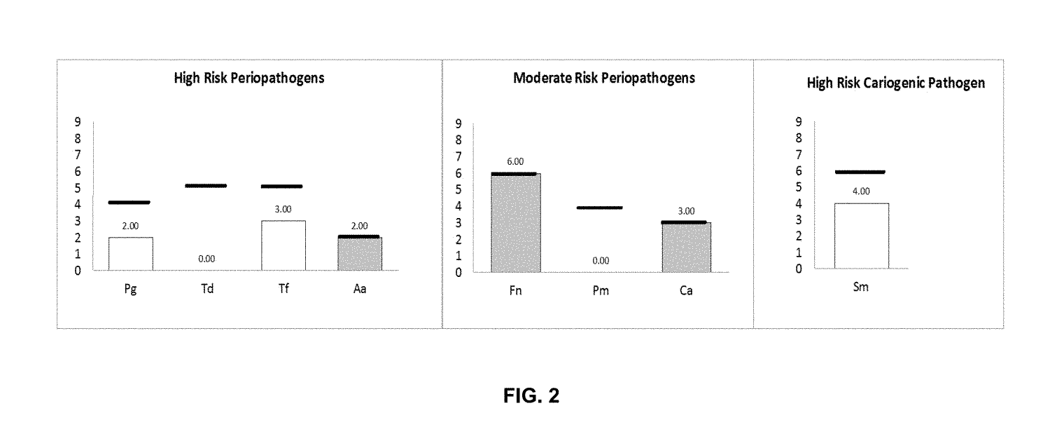

The patient information and test results are provided in Table 6 and FIG. 2.

TABLE 6
Patient and Test information

| Patient Information | Test Information |
|---|---|
| Name of Patient: n/a (Patient B) | Ordered By: n/a |
| Date of Birth: n/a | Clinic Name: n/a |
| Gender: n/a | Address: n/a |
| Medical/Dental concerns: implant placed, | State/Province/Country: n/a |
| dental decay, candidiasis | |
| Maximum Pocket Depth: 3 mm | Postal or Zip Code: n/a |
| Periodontal disease: mild, localized | Date Sample Collected: |
| Allergies: none | Date Sample Rec'd: |
| Antibiotic history: not used within | Report Date: |
| the last 3 months | |

FIG. 2 illustrates the levels of target pathogens from the sample of Patient B. In summary, high and moderate risk pathogens were detected for Patient B. The detailed report was as follows:
Periodontal risk: High-risk periodontal pathogen A. actinomycetemcomitans was found at threshold. T. forsythia and P. gingivalis were at normal level and T. denticola was not present. Moderate-risk pathogen F. nucleatum was detected at the threshold. P. micros was not present.
Caries risk: S. mutans was detected within normal limits, indicating that other cariogenic pathogens may be responsible for the reported dental decay.
Candidiasis: C. albicans was found elevated, which likely contributes to the reported oral yeast infection.

Treatment was selected and recommended based on medical history, periodontal charting, and the above report.

c. Case C

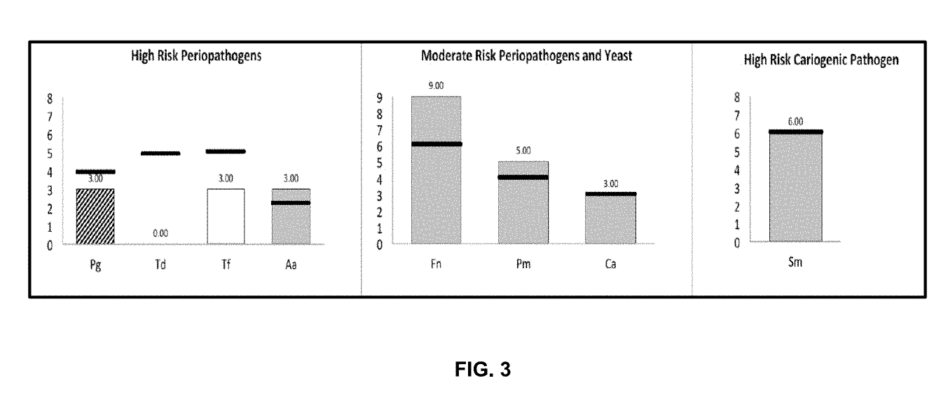

The test results of Case C are shown in FIG. 3, which illustrates the levels of target pathogens from the sample of Patient C.

In summary, high and moderate risk pathogens were detected for Patient C. The detailed report was as follows:
Periodontal risk: High-risk periodontal pathogen A. actinomycetemcomitans was identified above the normal threshold. P. gingivalis was at monitor level and T. forsythia was within normal range. T. denticola was not detected. Moderate-risk pathogens F. nucleatum and P. micros were found above normal levels.
Caries risk: S. mutans was detected above the normal range. That may increase the risk for development of dental caries.
Candidiasis: C. albicans was found at higher numbers than normal level and that may predispose to oral yeast infection.

Treatment was selected and recommended based on medical history, periodontal charting, and the above report. The specific treatment recommendations for Patient C were as follows:
1. There are substantial periodontal pathogens present in the sample we received. Most of the pathogens in this report are susceptible to metronidazole but the Aa responds best to amoxicillin. Therefore, it is recommended that Patient C use the preparation FM4+Amox 3 times a day until completed, rinsing vigorously for 30 seconds and then spitting the mixture out.
2. Tongue cleaning is recommended prior to rinsing to allow for closer contact with the antibiotic rinse. Patient C should use Soft-Picks coated with FM4+Amox rinse to clean between the teeth. This is an important step that will help to break up the biofilm and also move particles into the sulcus.
3. After completing the antibiotic rinse, Patient C should rinse with Cx620 twice a day for two weeks and continue to use Soft-Picks twice a day to clean between the teeth, but this time wet with Cx620. The Soft-Picks will clean interdentally and also release some chlorhexidine into the sulcus.
4. At the re-evaluation appointment, use disclosing solution to identify biofilm, especially at the gingival margin and in between the teeth as this will help Patient C to focus on those areas when brushing.
5. Maintenance should consist of effective interdental cleaning and a daily mouthwash, such as OraVital CDLx or SmartMouth ACF (Advanced Clinical Formula).

### Example 3—Preparation of Oral Rinses and Cream

Various oral rinses and creams were prepared by commonly-known methods and also as described in Canadian Patent Application No. 2,458,219 A1, published on Mar. 6, 2003, and in “Treatment of oral malodor and periodontal disease using an antibiotic rinse”, Southward and Bosy, General Dentistry, July 2013, pp. 41-45.

Table 7 shows the compositions and physical characteristics of the various oral formulations prepared.

TABLE 7
Compositions of Oral Rinses and Cream and Characteristics Thereof

| Formulations | Compositions | | | Characteristics |
|---|---|---|---|---|
| Formulation | FM2 Rinse | 150 ml | | Consistency of this |
| I | Metronidazole: | 3 g | | formulation is liquid, |
| | Nystatin Suspension: | 100 ml (100,000 u/m) | | but it is cloudy in |
| | Distilled water: | 45 ml | | appearance. |
| | Additives: | Flavouring and Stevia for | | |
| | | sweetening are added. | | |
| Formulation | FM3 Cream | 30 g | 15 g | Consistency of this |
| II | Metronidazole: | 6 g | 3 g | formulation is similar |
| | Nystatin Cream, | 18 g | 9 g | to face cream, but it |
| | 100,000 IU: | | | may have a very |
| | Glaxal Base: | 6 g | 3 g | slight grainy feel |
| | | | | depending on the |
| | | | | metronidazole that is |
| | | | | being used. Usually, |
| | | | | the formulation has a |
| | | | | slight yellow colour. |

| | | | |
|---|---|---|---|
| Formulation | FM4 Rinse | 150 ml | Consistency of this |
| III | Metronidazole USP: | 4.5 g | formulation is liquid, |
| | Nystatin 5873 IU/mg | 1.7064 g | but it is cloudy in |
| | Powder: | | appearance. |
| | Distilled Water: | 150 ml | |
| | Additives: | Flavouring and Stevia for | |
| | | sweetening are added, | |
| Formulation | FM4-Amox Rinse | 150 ml | Consistency of this |
| IV | Metronidazole: | 4.5 g | formulation is liquid |
| | Nystatin Powder: | 1.7064 g | but it is cloudy in |
| | Amoxicillin: | 1.5 g | appearance. |
| | Distilled Water: | 150 ml | Refrigeration is |
| | Additives: | Flavouring and Stevia for | required. |
| | | sweetening are added. | |
| Formulation | TET2 Rinse | 150 ml | Colour of this |
| V | Tetracycline: | 3 g | formulation may |
| | Diphenhydramine: | 200 mg | range from yellow to |
| | Nystatin suspension: | 100 ml | brown depending on |
| | Distilled water: | 45 ml | the tetracycline that |
| | | | is used. The |
| | | | formulation has very |
| | | | little grain in |
| | | | consistency. |

A metronidazole-nystatin mixture was prepared for use as an oral rinse to treat halitosis, gingivitis, tonsillitis, and/or mild to moderate periodontal disease. Tablets of metronidazole (APO-Metronidazole™, Apotex Corp.) were crushed and grounded, the larger particles filtered out, and the fine particles mixed with nystatin suspension or nystatin powder (Nilstat™, Alimed or PMS PharmaScience) in distilled water. Metronidazole and nystatin are sparingly soluble or practically insoluble in water and form a suspension or dispersion.

A metronidazole-nystatin-amoxicillin mixture was prepared for use as an oral rinse to treat severe and refractory periodontal disease. Amoxicillin was added to the metronidazole-nystatin mixture prepared as described above.

A tetracycline-diphenhydramine-nystatin mixture was prepared for use as an oral rinse to treat halitosis, mild to moderate gingivitis, and/or caries. Tetracycline, diphenhydramine, and nystatin suspension were mixed and formulated in distilled water.

### Example 4—Clinical Study

A clinical study was carried out to determine the effectiveness of an antibiotic antifungal rinse preparation, containing metronidazole and nystatin, in decreasing oral malodor and periodontal disease for individuals whose chief complaint was halitosis (see “Treatment of oral malodor and periodontal disease using an antibiotic rinse”, Southward and Bosy, General Dentistry, July 2013, pp. 41-45).

Of the 1000 patient charts sent electronically to the University of Michigan for analysis, 649 participants were selected based on complete pre- and post-treatment data. Tongue base, tongue dorsum, and proximal areas of the dentition were evaluated for odors. Biofilm samples from these areas were taken from each patient. A Gram stain analysis provided morphological information on the microorganisms present in the oral cavity. In addition, the microbiology of the teeth and tongue was tested with a BANA strip for red complex clusters. Six-point periodontal probing was used to assess pocket depth and bleeding points.

Treatment was based on odor levels, the BANA test, and the results of the microbiology samples. Treatment initially consisted of rinsing with chlorhexidine 0.2% for 2 weeks, twice daily. Patients complained that, although their breath problems had decreased in intensity, some of the odor and bad taste remained, proving that the use of chlorhexidine alone was insufficient to completely reduce oral malodor. To improve treatment response, systemic metronidazole and clindamycin were used to treat the patients, followed by the 0.2% chlorhexidine. However, systemic side effects and the use by patients of other pharmaceutical medications complicated this approach. Thus, another method had to be designed to successfully treat the chief complaint of halitosis.

The treatment chosen was the metronidazole-nystatin rinse prepared as described in Example 3. Three times a day, patients dispensed a “capful” (2 ml) of this mixture, swished and gargled for 30 seconds, and expectorated the contents. Once per day, they flossed immediately after rinsing. After each rinse with antibiotic mixture, patients were to abstain from eating or drinking for 30 minutes. After 2 weeks of rinsing, patients returned to the clinic for an evaluation of treatment. Breath odor measurements and periodontal measurements were repeated, and compared with those taken at the initial appointment. Microbiology samples of the tongue base, tongue dorsum, and teeth were taken, analyzed, and compared with the pre-treatment samples. Patients were then placed on chlorhexidine 0.2% for 2 more weeks, followed by routine rinsing with non-prescription mouthwashes that the patient selected.

As shown in Table 8, the post-treatment reduction of oral malodor was 79%. As shown in Table 9, the difference in bleeding points pre- and post-treatment was 87%. There was a decrease in the number of teeth with 6 and 7 mm pockets by 76% and teeth with 5 mm pockets decreased by 84%. All were highly significant (P<0.0001).

While the foregoing invention has been described in some detail for purposes of clarity and understanding, it will be appreciated by one skilled in the art, from a reading of the disclosure that various changes in form and detail can be made without departing from the true scope of the invention in the appended claims.

What is claimed is:

1. A method of selecting a treatment for an oral infection in a patient, the method comprising:
using nucleic-acid-based analysis to analyze samples of saliva, tongue biofilm, and interproximal biofilm collected from the patient's oral cavity, to determine a pathogen profile of the oral cavity; and
selecting an antibiotic rinse as the treatment, based on the oral infection reported in the pathogen profile.

2. The method of claim 1, wherein the oral infection is a bacterial or fungal infection.

3. The method of claim 2, wherein the bacterial infection comprises infection of one or more of Aggregatibacter actinomycetemcomitans, Porphyromonas gingivalis, Treponema denticola, Tannerella forsythia, Fusobacterium nuceatum, Peptostreptococcus micros, Prevotella intermedia, Streptococcus mutans, Capnocytophaga species, Streptococcus viridans, Streptococcus pyogenes, Campylobacter rectus, Eubacterium nodatum, and Eikenella corrodens.

4. The method of claim 2, wherein the fungal infection comprises a Candida albicans infection.

5. The method of claim 2, wherein the oral infection comprises one or more of periodontal disease, dental caries, candidiasis, and halitosis.

6. The method of claim 1, wherein the nucleic-acid-based analysis comprises a polymerase chain reaction (PCR) assay.

7. The method of claim 6, wherein the PCR assay comprises a qPCR assay.

8. The method of claim 1, wherein the saliva is collected under the tongue, around the mandibular teeth, and/or around the palate side of the maxillary teeth.

9. The method of claim 1, wherein the tongue biofilm is collected around the tongue base and/or tongue dorsum.

10. The method of claim 1, wherein the interproximal biofilm is collected by inserting paper points into gingival sulci and/or periodontal pockets in at least 5 locations in the oral cavity.

11. The method of claim 1, wherein the pathogen profile reports counts of pre-selected target pathogens.

12. The method of claim 11, wherein the pre-selected target pathogens comprise one or more of Aggregatibacter actinomycetemcomitans, Porphyromonas gingivalis, Treponema denticola, Tannerella forsythia, Fusobacterium nuceatum eptostreptococcus micros, Prevotella intermedia, Candida albicans, Streptococcus mutans, Capnocytophaga species, Streptococcus viridans, Streptococcus pyogenes, Campylobacter rectus, Eubacterium nodatum, and Eikenella corrodens.

13. The method of claim 11, wherein selecting an antibiotic rinse comprises evaluating the pathogen profile with at least one of the patient's habits, health history, and symptoms.

14. The method of claim 11, wherein the pathogen profile reports presence of Aggregatibacter actinomycetemcomitans above a pre-determined threshold and the selected antibiotic rinse comprises amoxicillin, metronidazole, and nystatin.

15. The method of claim 11, wherein the pathogen profile reports presence of Aggregatibacter actinomycetemcomitans at or below a pre-determined threshold and the selected antibiotic rinse comprises metronidazole and nystatin.

16. The method of claim 11, wherein the target pathogens comprise Aggregatibacter actinomycetemcomitans, Porphyromonas gingivalis, Treponema denticola, and Tannerella forsythia, and further comprise at least one of Fusobacterium nuceatum, Peptostreptococcus micros, Prevotella intermedia, Candida albicans, Streptococcus mutans, and Capnocytophaga species; and
wherein the pathogen profile reports presence of three or more of the target pathogens above their respective pre-determined thresholds; and
the selected antibiotic rinse comprises amoxicillin.

17. The method of claim 12, wherein the pathogen profile reports presence of two or more of the target pathogens above their respective pre-determined thresholds and the selected antibiotic rinse comprises amoxicillin.

18. A method for treating an oral infection in a patient, comprising:
using nucleic-acid-based analysis to analyze samples of saliva, tongue biofilm, and interproximal biofilm collected from the patient's oral cavity, to determine a pathogen profile of the oral cavity; and
selecting an antibiotic rinse as treatment, based on the oral infection reported in the pathogen profile.

19. The method of claim 18, further comprising administering the selected antibiotic rinse as the treatment to the patient.

20. A kit for use in selecting a treatment for an oral infection in a patient, the kit comprising:
primers specific to nucleic acid sequences of pre-selected target pathogens, for use in conducting a polymerase chain reaction (PCR) assay to analyze samples of saliva, tongue biofilm, and interproximal biofilm collected from the patient's oral cavity and determining a pathogen profile of the oral cavity; and
instructions to select an antibiotic rinse as the treatment based on the oral infection reported in the pathogen profile.
